SEQUENCE LISTING (14 sequences)

SEQ ID NO: 1 - protein, 930 aa, Homo sapiens
MKPPRPVRTCSKVLVLLSLLAIHQTTTAEKNGIDIYSLTVDSRVSSRFAHTVVTSRVVNRANTVQEATFQMELPKKAFITNFSMNIDGMTYPGIIKEKAEAQAQYSAAVAKGKNAGLVKATGRNMEQFQVSVSVAPNAKITFELVYEELLKRRLGVYELLLKVRPQQLVKHLQMDIHIFEPQGISFLETESTFMTNQLVDALTTWQNKTKAHIRFKPTLSQQQKSPEQQETVLDGNLIIRYDVDRAISGGSIQIENGYFVHYFAPEGLTTMPKNVVFVIDKSGSMSGRKIQQTREALIKILDDLSPRDQFNLIVFSTEATQWRPSLVPASAENVNKARSFAAGIQALGGTNINDAMLMAVQLLDSSNQEERLPEGSVSLIILLTDGDPTVGETNPRSIQNNVREAVSGRYSLFCLGFGFDVSYAFLEKLALDNGGLARRIHEDSDSALQLQDFYQEVANPLLTAVTFEYPSNAVEEVTQNNFRLLFKGSEMVVAGKLQDRGPDVLTATVSGKLPTQNITFQTESSVAEQEAEFQSPKYIFHNFMERLWAYLTIQQLLEQTVSASDADQQALRNQALNLSLAYSFVTPLTSMVVTKPDDQEQSQVAEKPMEGESRNRNVHSGSTFFKYYLQGAKIPKPEASFSPRRGWNRQAGAAGSRMNFRPGVLSSRQLGLPGPPDVPDHAAYHPFRRLAILPASAPPATSNPDPAVSRVMNMKIEETTMTTQTPAPIQAPSAILPLPGQSVERLCVDPRHRQGPVNLLSDPEQGVEVTGQYEREKAGFSWIEVTFKNPLVWVHASPEHVVVTRNRRSSAYKWKETLFSVMPGLKMTMDKTGLLLLSDPDKVTIGLLFWDGRGEGLRLLLRDTDRFSSHVGGTLGQFYQEVLWGSPAASDDGRRTLRVQGNDHSATRERRLDYQEGPPGVEISCWSVEL

SEQ ID NO: 2 - protein, 242 aa, Homo sapiens
RLAILPASAPPATSNPDPAVSRVMNMKIEETTMTTQTPAPIQAPSAILPLPGQSVERLCVDPRHRQGPVNLLSDPEQGVEVTGQYEREKAGFSWIEVTFKNPLVWVHASPEHVVVTRNRRSSAYKWKETLFSVMPGLKMTMDKTGLLLLSDPDKVTIGLLFWDGRGEGLRLLLRDTDRFSSHVGGTLGQFYQEVLWGSPAASDDGRRTLRVQGNDHSATRERRLDYQEGPPGVEISCWSVEL

SEQ ID NO: 3 - protein, 256 aa, Homo sapiens
RLAILPASAPPATSNPDPAVSRVMNMKIEETTMTTQTPACPSCSRSRAPAVPAPIQAPSAILPLPGQSVERLCVDPRHRQGPVNLLSDPEQGVEVTGQYEREKAGFSWIEVTFKNPLVWVHASPEHVVVTRNRRSSAYKWKETLFSVMPGLKMTMDKTGLLLLSDPDKVTIGLLFWDGRGEGLRLLLRDTDRFSSHVGGTLGQFYQEVLWGSPAASDDGRRTLRVQGNDHSATRERRLDYQEGPPGVEISCWSVEL

SEQ ID NO: 4 - protein, 313 aa, Homo sapiens
MELDRAVGVLGAATLLLSFLGMAWALQAADTCPEVKMVGLEGSDKLTILRGCPGLPGAPGDKGEAGTNGKRGERGPPGPPGKAGPPGPNGAPGEPQPCLTGPRTCKDLLDRGHFLSGWHTIYLPDCRPLTVLCDMDTDGGGWTVFQRRVDGSVDFYRDWATYKQGFGSRLGEFWLGNDNIHALTAQGTSELRVDLVDFEDNYQFAKYRSFKVADEAEKYNLVLGAFVEGSAGDSLTFHNNQSFSTKDQDNDLNTGNCAVMFQGAWWYKNCHVSNLNGRYLRGTHGSFANGINWKSGKGYNYSYKVSEMKVRPA

SEQ ID NO: 5 - protein, 299 aa, Homo sapiens
AKVEQAVETEPEPELRQQTEWQSGQRWELALGRFWDYLRWVQTLSEQVQEELLSSQVTQELRALMDETMKELKAYKSELEEQLTPVAEETRARLSKELQTAQARLGADMEDVCGRLVQYRGEVQAMLGQSTEELRVRLASHLRKLRKRLLRDPDDLQKRAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 6 - protein, 249 aa, Homo sapiens
RHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 7 - protein, 352 aa, Homo sapiens
MRSLGALLLLLSACLAVSAGPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPRVRRAVLPQEEEGSGGGQLVTEVTKKEDSCQLGYSAGPCMGMTSRYFYNGTSMACETFQYGGCMGNGNNFVTEKECLQTCRTVAACNLPIVRGPCRAFIQLWAFDAVKGKCVLFPYGGCQGNGNKFYSEKECREYCGVPGDGDEELLRFSN

SEQ ID NO: 8 - protein, 181 aa, Homo sapiens
GPVPTPPDNIQVQENFNISRIYGKWYNLAIGSTCPLKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPR

SEQ ID NO: 9 - protein, 1744 aa, Homo sapiens [sequence omitted]

SEQ ID NO: 10 - protein, 291 aa, Homo sapiens
EAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 11 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 12 - protein, 609 aa, Homo sapiens [sequence omitted]

SEQ ID NO: 13 - protein, 399 aa, Homo sapiens
DEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 14 - protein, 232 aa, Homo sapiens [sequence omitted]

(19) United States
(12) Patent Application Publication
(10) Pub. No.: US 2009/0035801 A1
(43) Pub. Date: Feb. 5, 2009

(54) Twelve (12) protein biomarkers for diagnosis and early detection of breast cancer

(75) Inventors: Ira Leonard Goldknopf, The Woodlands, TX (US); Essam Ahmed Sheta, The Woodlands, TX (US); Jennifer Kathleen Bryson, The Woodlands, TX (US); Alan B. Hollingsworth, Oklahoma City, OK (US)

Correspondence Address: Power3 Medical Products, Inc.;Steven B. Rash, 3400 Research Forest Drive, The Woodlands, TX, 77381, US

(73) Assignee: Power3 Medical Products, Inc., The Woodlands, TX (US)

(21) Appl. No.: 11/635,281

(22) Filed: Dec. 7, 2006

Related U.S. Application Data
(60) Provisional application No. 60/754,441, filed on Dec. 27, 2005.
(60) Provisional application No. 60/834,649, filed on Aug. 1, 2006.

(51) Int. Cl. C12Q 1/02

(52) U.S. Cl. 435/29

(57) ABSTRACT

The invention relates to 12 identified protein biomarkers for diagnosis, determination of disease severity, and therapeutic response monitoring of patients with breast cancer. The method is based on the use of 2-dimensional (2D) gel electrophoresis to separate the complex mixture of proteins found in blood serum, the quantitation of up to 12 protein biomarkers, and statistical analysis of the concentration of the protein biomarkers.

### CROSS REFERENCE TO RELATED APPLICATIONS

This application claims priority to U.S. Provisional patent application 60/754,441 on Dec. 27, 2005 and entitled “Multivariate biostatistics of 12 Blood Serum Protein Biomarkers Distinguishes Women with Breast Cancer, Benign Breast Disease, and Normal Controls. Role of: Inter-Alpha-Trypsin Inhibitor Heavy Chain Like Protein Variants, Lectin P35, Apolipoprotein E3, Apolipoprotein A1, Alpha-a-microglobulin, and Apolipoprotein J in Tests” by inventors Ira L. Goldknopf et al. It also claims priority to U.S. Provisional patent application 60/834,649 filed on Aug. 1, 2006 and entitled “Multivariate biostatistics of 12 Blood Serum Protein Biomarkers Distinguishes Women with Breast Cancer, Benign Breast Disease, and Normal Controls. Role of: Inter-Alpha-Trypsin Inhibitor Heavy Chain Like Protein Variants, Lectin P35, Apolipoprotein E3, Apolipoprotein A1, Alpha-1-microglobulin, Complement component C4A, and Transferrin in the Tests” by inventors Ira L. Goldknopf et al.

### BACKGROUND OF THE INVENTION

1. Field of the Invention

This invention relates to twelve (12) protein biomarkers of breast cancer. More specifically, the invention relates to 12 protein biomarkers in blood serum that can be used in diagnosis, determination of disease severity, and monitoring of therapeutic response of patients with breast cancer. The method is based on the use of two-dimensional (2D) gel electrophoresis to separate the complex mixture of proteins found in blood serum, the quantitation of 12 identified protein spots, and statistical analysis, to distinguish patients with breast cancer from patients with benign breast disease or abnormalities, and from normal women, for the purpose of diagnosis, for determination of disease severity, and for treatment response monitoring.

2. Description of the Related Art

There is an urgent need for objective diagnostic tests to detect breast cancer in its earliest stages. By the time a patient is diagnosed with breast cancer by mammography and subsequent biopsy, the patient has had the disease for an average 6-10 years (Spratt, J. S. et al. 1986, Cancer Research 46, 970-974, A. Hollingsworth, personal communication Dec. 2, 2004 re Spratt et al). In addition, when mammography is the only screening tool utilized, it has to be remembered that sensitivity here is only 70% overall even with digital technology, and mammography was recently found in a major trial to have a mere 41% sensitivity when a 15-month follow-up period was used to define false-negatives. (Pisano et al. 2005, N Engl J Med 353, 1773-1783). _{[ABH1]}MRI detects breast cancer earlier, and with much greater sensitivity, than mammograms (Hollingsworth, A. B. et al. 2003, J OK. St. Med. Assoc. 96, Hollingsworth A. B. et al. 2004 Amer. J. Surgery 187 349-362). Genetic mutational tests (BRCA 1 and 2 genes) detect genetic disposition of breast cancer risk, but aggressive screening, usually with breast MRI, is chosen more often than preventive mastectomy by patients who tests BRCA-positive Hollingsworth A. B. et al. 2004; Robson, M. E. et al. 2004, JAMA 292, 1368-1370). Whereas the need for imaging of breast tumors will always be required for localization and treatment. A sensitive early detection screening test with cost comparable to mammograms is needed to justify the high cost and insurance reimbursement for auxiliary imaging with ultrasound and/or MRI._{[ABH2]}

There has been a tremendous interest in the potential ability of proteomic technology to fulfill the unmet needs of effective strategies for early diagnosis of cancer (Alaiya, A. et al. 2005, J. Proteome Res. 4: 1213-1222) with a special emphasis on cancer detection in biological fluids from patients, including ovarian cancer (Emmanuel F. Petricoin, A. M. Ardekani, B. A. Hitt et al. 2002, Lancet 359: 572-577) and breast cancer (Paweletz C. P. et al 2001, Dis. Markers 17: 301-307; Henry M. Kuerer, H. M. et al. 2002, Cancer 95: 2276-2282). Proteomics is a new field of medical research wherein proteins are identified and linked to biological functions, including roles in a variety of disease states. With the completion of the mapping of the human genome, the identification of unique gene products, or proteins, has increased exponentially. In addition, molecular diagnostic testing for the presence of proteins already known to be involved in certain biological functions has progressed from research applications alone to use in disease screening and diagnosis for clinicians. However, proteomic testing for diagnostic purposes remains in its infancy.

Detection of abnormalities in the genome of an individual can reveal the risk or potential risk for individuals to develop a disease. The transition from gene based risk to emergence of disease can be characterized as an expression of genomic abnormalities in the proteome. In fact, whether arising from genetic, environmental, or other factors, the appearance of abnormalities in the proteome signals the beginning of the process of cascading effects that can result in the deterioration of the health of the patient. Therefore, detection of proteomic abnormalities at an early stage is desired in order to allow for detection of disease processes either before the disease is established or in its earliest stages where treatment may be more effective.

Recent progress using a novel form of mass spectrometry called surface enhanced laser desorption and ionization time of flight (SELDI-TOF) for the testing of ovarian cancer and Alzheimer's disease has led to an increased interest in proteomics as a diagnostic tool (Petrocoin, E. F. et al. 2002. Lancet 359:572-577, Lewczuk, P. et al. 2004. Biol. Psychiatry 55:524-530). Furthermore, proteomics has been applied to the study of breast cancer through use of 2D gel electrophoresis and image analysis to study the development and progression of breast carcinoma in patients' breast ductal fluid specimens ((Kuerer, H. M. et al. 2002. Cancer 95:2276-2282) and in plasma (Goufman, et al. 2006. Biochemistry 2006, 71(4):354-60). In the case of breast cancer, breast ductal fluid specimens were used to identify distinct protein expression patterns in bilateral matched pair ductal fluid samples of women with unilateral invasive breast carcinoma (Kuerer, H. M. et al. 2002).

Detection of biomarkers is an active field of research. For example, U.S. Pat. No. 5,958,785 discloses a biomarker for detecting long-term or chronic alcohol consumption. The biomarker disclosed is a single biomarker and is identified as an alcohol-specific ethanol glycoconjugate. U.S. Pat. No. 6,124,108 discloses a biomarker for mustard chemical injury. The biomarker is a specific protein band detected through gel electrophoresis and the patent describes use of the biomarker to raise protective antibodies or in a kit to identify the presence or absence of the biomarker in individuals who may have been exposed to mustard poisoning. U.S. Pat. No. 6,326,209 B 1 discloses measurement of total urinary 17 ketosteroid-sulfates as biomarkers of biological age. U.S. Pat. No. 6,693,177 B1 discloses a process for preparation of a single biomarker specific for O-acetylated sialic acid and useful for diagnosis and outcome monitoring in patients with lymphoblastic leukemia.

Two-dimensional (2D) gel electrophoresis has been used in research laboratories for biomarker discovery since the 1970's (Margolis J. et al. 1969, Nature. 1969221: 1056-1057; Orrick, L. R. et al. 1973; Proc Nat'l Acad. Sci. USA. 70: 1316-1320; Goldknopf, I. L. et al. 1975, J Biol Chem. 250: 7182-7187; Goldknopf, I. L. et al. 1977, Proc Nat'l Acad Sci USA. 74: 5492-5495; O'Farrell, P. H. 1975, J. Biol. Chem. 250: 4007-4021; Anderson, L. 1977, Proc Nat'l Aced Sci USA. 74: 864-868; Klose, J. 1975, Human Genetic. 26: 231-243). The advent of much faster identification of proteins spots by in-gel digestion and mass spectroscopy ushered in the accelerated development of proteomic science through large-scale application of these techniques (Aebersold R. 2003, Nature, 422: 198-207; Kuruma, H. et al. 2004, Prostate Cancer and Prostatic Disease 1: 1-8; Kuncewicz, T. et al. 2003, Molecular & Cellular Proteomics 2: 156-163). With the advent of bioinformatics, progression of proteomics towards diagnostics and personalized medicine has become feasible (White, C. N. et al. 2004 Clinical Biochemistry, 37: 636-641; Anderson N. L. et al. 2002, Molecular & Cellular Proteomics 1:845-867). Clinical proteomics is maturing fast into a powerful approach for comprehensive analyses of disease mechanisms and disease markers (Kuruma, H. et al. 2004; Sheta, E. A. et al. 2006, Expert Rev. Proteomics 3: 45-62). We have recently applied 2D gel proteomics of human serum combined with discriminant biostatistics to the differential diagnosis of neurodegenerative diseases (Goldknopf, I. L. et al. 2006, Biochem. Biophys. Res. Commun. 342: 1034-1039; Sheta, E. A. et al. 2006). In the present invention, we use the same approach to monitor the concentrations of 12 protein biomarkers, resolved and quantitated by 2D gel electrophoresis of blood serum, to distinguish between patients who have been diagnosed with increasingly severe breast cancer, with benign breast disease, and with no breast abnormalities as normal controls.

### SUMMARY OF THE INVENTION

The present invention relates to 12 protein biomarkers in blood serum for screening, diagnosis, determination of disease severity, and monitoring response to treatment, of breast cancer. More specifically, the present invention consists of up to 12 protein biomarkers in blood and their use in diagnostic assays for differentiating between patients of breast cancer, patients having benign breast disease or abnormalities, and normal individuals. The method comprises collecting a biological sample from patients having biopsy confirmed and histological staged breast cancer, patients having benign breast disease or abnormalities, and patients having no evidence of breast disease or breast abnormality, then determining the concentrations of up to 12 protein biomarkers identified as related to breast cancer. Patients are then sorted into these respective groupings based on a statistical analysis of the concentration in blood serum of up to 12 protein biomarkers.

One aspect of the present invention is the use of up to 12 biomarkers for screening a patient for breast cancer. The method includes: collecting a biological sample from a patient, determining the concentrations of up to 12 protein biomarkers identified as related to breast cancer, and determining whether or not the patient has breast cancer, based on a statistical analysis of the concentration in blood serum of one or more of the selected 12 protein biomarkers. This aspect of the invention can be used as an early blood screen in patients to complement mammography, such that a negative mammogram but a positive blood test would signal the need for more sensitive imaging such as breast MRI. In the case of an equivocal mammogram, the predictive power of a blood test would help the radiologist to decide whether or not to proceed with biopsy. Another aspect of the present invention is the use of up to 12 protein biomarkers for determining the severity of breast cancer and/or monitoring the response to treatment of a patient. The method includes: collecting a biological sample from a patient, determining the concentrations of up to 12 protein biomarkers identified as related to breast cancer, and determining the severity of breast cancer and/or response of the patient to treatment based on the concentrations in blood serum of up to 12 protein biomarkers. For example, this aspect of the invention can be used to help the oncologist make decisions about specific chemotherapeutic and/or antihormonal regimens, or newer biologic weapons, and to monitor the response to treatment.

Another aspect of the present invention is the use of up to 12 biomarkers for determining the biological mechanism of disease of a patient and/or the drug target of the patient for treatment of breast cancer. The method includes: collecting a biological sample from a patient, determining the concentrations of up to 12 protein biomarkers identified as related to breast cancer, and determining the mechanism of disease active in the patient and/or identifying the drug target appropriate for treatment of the patient, based on the concentration in blood serum of up to 12 protein biomarkers.

The foregoing has outlined rather broadly several aspects of the present invention in order that the detailed description of the invention that follows may be better understood. Additional features and advantages of the invention will be described hereinafter which form the subject of the claims of the invention. It should be appreciated by those skilled in the art that the conception and the specific embodiments disclosed might be readily utilized as a basis for modifying or redesigning the methods for carrying out the same purposes as the invention. It should be realized by those skilled invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1: A representative 2D gel electrophoretic image of human serum proteins with the positions of the 12 protein biomarker spots indicated by circles and numbers.

FIG. 2: Box and whisker plot (constructed using Analyze-it software for Microsoft XL) of blood serum concentrations (PPM) of the breast cancer biomarkers depicted in FIG. 1 from patients with breast cancer, benign breast abnormalities or disease, and normal controls subjects.

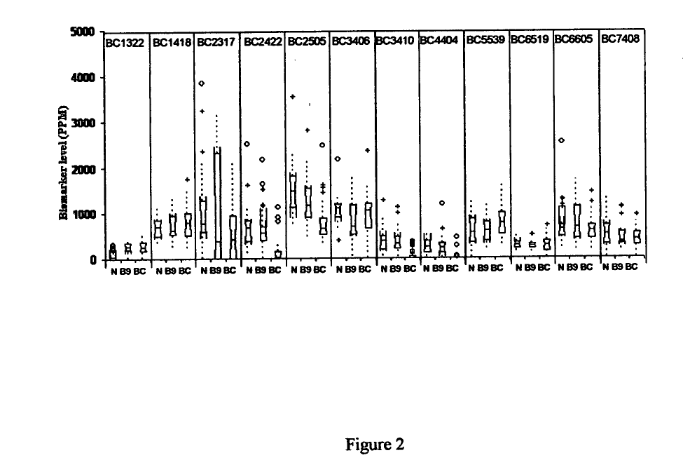

FIG. 3: Box and whisker plot (constructed using Analyze-it software for Microsoft XL) of blood serum concentrations of four electrophoretic isoforms of the biomarker Inter-a-Trypsin Heavy Chain Related (H4) Protein, 35 KD, processing product (ITIHRP), corresponds to biomarker spots #2422, 2505, 3410, and 4404) in normal control subjects (N), patients with benign breast abnormalities or disease (B9), and breast cancer patients (BC), divided into earlier Stages (BC 0-I) and later stages (BC II-III).

FIG. 4: Box and whisker plot of total blood serum concentrations (PPM) of the sum of all four electrophoretic isoforms of Inter-a-Trypsin Heavy Chain (H4) Related Protein 35 KD processing product (biomarker spots #2422+2505+3410+4404) in normal control subjects (N), patients with benign breast abnormalities or disease (B9), earlier stage (BC 0-I) and later stage (BC II-III) breast cancer patients. The data indicates the progressive drop in concentration between normal (N) and benign (B9), versus breast cancer stages.

FIG. 5: Amino acid sequence homologies between the 3 heavy chain variants of inter-α-trypsin inhibitor heavy chain isoforms (HC1, HC2, HC3) and the inter-a-trypsin inhibitor heavy chain H4 (HC4, PK-120) related protein, with its 35 KD processing product (biomarker spots #2422, 2505, 3410, and 4404). As shown PK-120 has only limited amino acid sequence homology to the amino acid sequences of the corresponding regions of the three inter-α-trypsin inhibitor heavy chain (HC1, HC2, HC3) isoforms, none of which correspond to biomarker spots 2422, 2505, 3410, and 4404.

FIG. 6: Box and whisker plot of blood serum concentrations (PPM) of biomarker spot #1322, an immunoglobulin lambda (λ) light chain, in normal control subjects, patients with benign breast abnormalities, and breast cancer patients. Data indicate the significant rise in concentration of this biomarker in patients with benign breast abnormalities (B9) and/or breast cancer early (BC 0-I) and late (BC II-III) stages, compared to normal subjects.

FIG. 7: Box and whisker plot of blood serum concentrations (PPM) of biomarker spot #1418, alpha-1-microglobulin. Data indicate the significant rise in concentration of this biomarker, in early stage (BC 0-I) breast cancer patients.

FIG. 8: Box and whisker plot of blood serum concentrations (PPM) of biomarker spot #2317, Apolipoprotein A-I. Data indicate the significant decline in concentration of this biomarker in later stage breast cancer (BC II-III) to an undetectable level (0 ppm). Analysis shows that 91% of BC II-III patients ( 10/11) have no detectable concentration of this biomarker in their serum compared to undetectable level in only 5% normal controls subjects ( 1/22) and 33% of patients with benign breast abnormalities or disease and 33% patients with earlier stage breast cancer BC 0-1 ( 8/24).

FIG. 9: Box and whisker plot of blood serum concentrations (PPM) of biomarker spot #3406, Apolipoprotein E3. Data indicate the significant drop in concentration in later stage breast cancer (BC II-III), when compared to normal subjects.

FIG. 10: Box and whisker plot of blood serum concentrations (PPM) of biomarker spot #6519, Lectin P35. Data shows significant decrease in concentration in blood serum of patients with breast cancer stage 0 (BC 0), compared to its level in normal subjects, benign patients and in breast cancer stages I and stages II-III.

Table I: The stages of breast cancer.

Table II: Protein standards for 2D gel electrophoresis.

Table III: Biomarker protein identifications by LC-MS/MS.

Table IV: Serum level (PPM) of the 4 isoforms of ITIHRP, spots #2422, 2505, 3410, 4404. Data shows significant lower serum levels (P<0.0001) in all 4 spots in breast cancer patients (early stages, BC0-I and late stages, BC II-III) compared to normal subjects and benign patients

Table V: Serum level (PPM) of immunoglobulin lambda light chain, spot #1322. Data shows significant higher serum levels (P<0.0001) of this biomarker in benign (B9) patients and breast cancer patients (early stages, BC0-I and late stages, BC II-III) compared to normal subjects.

Table VI: Serum level (PPM) of Alpha-1-microglobulin, spot #1418. Data shows significant higher serum levels (P<0.027) of this biomarker in early breast cancer stage patients (BC0-I) compared to normal subjects.

Table VII: Serum level (PPM) of Apolipoprotein A-I, spot #2317. Data shows significant lower serum levels of this biomarker in late stage breast cancer (BC II-III) patients compared to normal (N) subjects (P<0.0016), benign (B9) patients (P<0.026) and early breast cancer (BC 0-I) patients (P<0.015).

Table VIII: Serum level (PPM) of Apolipoprotein E3, spot #3406. Data shows significant lower serum levels of this biomarker in late stage breast cancer (BC II-III) patients compared to normal (N) subjects (P<0.0006) and its significantly higher serum level in early stage breast cancer (BC 0-I) patients compared to benign (B9) patients (P<0.002) and late stage breast cancer (BC II-III) patients (P<0.0001).

Table IX: Serum level (PPM) of Lectin P35, spot #6519. Data shows significant lower serum levels of this biomarker in early stage breast cancer (BC 0) patients compared to normal (N) subjects (P<0.0028), benign (B9) patients (P<0.0025), early stage breast cancer (BC I) patients (P<0.046) and late stage breast cancer (BC II-III) patients (P<0.0051).

Table X: Number of patients and percent classified into diagnosis by multi-variate quadratic and linear discriminant biostatistics using all 12 biomarker spot concentrations from a total of 98 individuals. Diagnoses illustrated in this example are 3-way (N vs. B9 vs. BC) illustrated in box A and B, and 2 way (Not Cancer (N-B9), combined normal (N) and benign (B9), compared to Cancer (BC0-III), combined all breast cancer stages patients, as illustrated in box C and D.

Table XI: Amino acid sequence of isoform 1 of inter-alpha-trypsin inhibitor heavy chain (H4) related protein (ITIHRP) parent protein, corresponds to biomarkers spots #2422, 2505, 3410, 4404, the 35 KD processing product.

Table XII: Amino acid sequences of isoforms 1 and 2 of inter-alpha-trypsin inhibitor heavy chain (H4) related protein (ITIHRP) (ITIH4), 35 KD processing products, and the tryptic peptide spans.

Table XIII: Sequence alignment of ITIHRP Isoform 1 and Isoform 2. Identical sequences are marked with stars while unmatched sequences are marked by dashes.

Table XIV: Amino acid sequence of Lectin P35 (spot #6519).

Table XV: Amino acid sequence of Apolipoprotein E3 (spot #3406).

Table XVI: Amino acid sequence of Apolipoprotein A1 (spot #2317).

Table XVII: Amino acid sequence of Alpha-1-microglobulin (spot #1418).

Table XVIII: Amino acid sequence of C4A including C4A? chain (spot #7408).

Table XIX: Amino acid sequence of parental protein Complement C4A

Table XX: Amino acid sequence of Transferrin (spot #6606).

Table XXI: Amino acid sequence of human serum Albumin (spot #5539).

Table XXII: Amino acid sequence of immunoglobulin lambda chain (spot #1322).

### DESCRIPTION OF THE PREFERRED EMBODIMENTS

The present invention is a diagnostic assay for differentiating between patients having breast cancer, patients with benign breast disease or abnormalities, and normal control individuals. The method is based on the use of two-dimensional (2D) gel electrophoresis to separate the complex mixture of proteins found in blood serum and the quantitation of a group of identified biomarkers to differentiate between patients having breast cancer, patients with benign breast disease or abnormalities, and normal control individuals.

In the context of the present invention breast cancer consists of biopsy confirmed and histological staged disease. The breast cancer may be from a plurality of stages, wherein staging is the process physicians use to assess the size and location of a patient's cancer. Identifying the cancer stage is one of the most important factors in selecting treatment options. In the present invention, the numerical stages of breast cancer are defined as:

TABLE I
Staging Breast Cancer

| | | | Metastasis |
|---|---|---|---|
| Stage | Tumor Size | Lymph Node Involvement | (Spread) |
| 0 | In situ (DCIS, LCIS) | No | No |
| I | Less than 2 cm | No | No |
| II | Between 2-5 cm | No or in same side of breast | No |
| III | More than 5 cm | Yes, on same side of breast | No |
| IV | Not applicable | Not applicable | Yes |

In the context of the present invention, the “protein expression profile” corresponds to the steady state level of the various proteins in biological samples that can be expressed quantitatively. These steady state levels are the result of the combination of all the factors that control protein concentration in a biological sample. These factors include but are not limited to: the rates of transcription of the genes encoding the hnRNAs; processing of the hnRNAs into mRNAs; The rates of splicing and the splicing variations during the processing of the hnRNAs into mRNAs which govern the relative amounts of the protein sequence isoforms; the rates of processing of the various mRNAs by 3′-polyadenylation and 5′-capping; the rates of transport of the mRNAs to the sites of protein synthesis; the rate of translation of the mRNA's into the corresponding proteins; the rates of protein post-translational modifications, including but not limited to phosphorylation, nitrosylation, methylation, acetylation, glycosylation, poly-ADP-ribosylation, ubiquitinylation, and conjugation with ubiquitin Like proteins; the rates of protein turnover via the ubiquitin-proteosome system and via proteolytic processing of the parent protein into various active and inactive subcomponents; the rates of intracellular transport of the proteins among compartments, such as but not limited to the nucleus, the lysosomes, golgi, the membrane, and the mitochondrion; the rates of secretion of the proteins into the interstitial space; the rates of secretion related protein processing; and the stability and rates of proteolytic processing and degradation of the proteins in the biological sample before and after the sample is taken from the patient. In the context of the present invention, a “biomarker” corresponds to a protein or protein fragment present in a biological sample from a patient, wherein the quantity of the biomarker in the biological sample provides information about whether the patient exhibits an altered biological state such as breast cancer of stages 0, I, II, III, IV, or benign breast disease or abnormalities.

A “control’ or “normal” sample is a sample, preferably a serum sample, taken from an individual with no known disease, particularly no known breast abnormalities.

The method of the present invention is based on the quantification of specified proteins. Preferably the proteins are separated and identified by 2D gel electrophoresis. In the past, this method has been considered highly specialized, labor intensive and non-reproducible.

Only recently with the advent of integrated supplies, robotics, and software combined with bioinformatics has progression of this proteomics technique in the direction of diagnostics become feasible. The promise and utility of 2D gel electrophoresis is based on its ability to detect changes in protein expression and to discriminate protein isoforms that arise due to variations in amino acid sequence and/or post-synthetic protein modifications such as phosphorylation, nitrosylation, ubiquitination, conjugation with ubiquitin-Like proteins, acetylation, and glycosylation.

These are important variables in cell regulatory processes involved in disease states. There are few comparable alternatives to 2D gels for tracking changes in protein expression patterns related to disease progression. The introduction of high sensitivity fluorescent staining, digital image processing and computerized image analysis has greatly amplified and simplified the detection of unique species and the quantification of proteins. By using known protein standards as landmarks within each gel run, computerized analysis can detect unique differences in protein expression and modifications between two samples from the same individual or between several individuals.

### Materials and Methods:

### Sample Collection and Preparation

Serum samples were prepared from blood acquired by venipuncture. The blood was allowed to clot at room temperature for 30-60 minutes, centrifuged at 1200×g for 15 minutes, and the separated serum was divided into aliquots, and frozen at −40° C. or below until shipment. Samples were shipped on dry ice and were delivered within 24 hours of shipping.

Once the serum samples were received, logged in, and assigned a sample number; they were further processed in preparation for 2D gel electrophoresis. All samples were stored at −80° C. or below. When the serum samples were removed from storage, they were placed on ice for thawing and kept on ice for further processing.

### Separation of Proteins in Patient Samples

The serum protein from patients and normal control subjects analyzed in the present invention were separated using 2D gel electrophoresis. Other various techniques known in the art for separating proteins can also be used. These other techniques include but are not limited to gel filtration chromatography, ion exchange chromatography, reverse phase chromatography, affinity chromatography, or any of the various centrifugation techniques well known in the art. In some cases, a combination of one or more chromatography or centrifugation steps may be combined via electrospray or nanospray with mass spectroscopy or tandem mass spectroscopy, or any protein separation technique that determines the pattern of proteins in a mixture either as a one-dimensional, two-dimensional, three-dimensional or multi-dimensional pattern or list of proteins present.

### Two Dimensional Gel Electrophoresis of Samples

Preferably the protein profiles of the present invention are obtained by subjecting biological samples to two-dimensional (2D) gel electrophoresis to separate the proteins in the biological sample into a two-dimensional array of protein spots.

Two-dimensional gel electrophoresis is a useful technique for separating complex mixtures of proteins and can be performed using a variety of methods known in the art (see, e.g., U.S. Pat. Nos. 5,534,121; 6,398,933; and 6,855,554).

Preferably, the first dimensional gel is an isoelectric focusing gel and the second dimension gel is a denaturing polyacrylamide gradient gel.

Proteins are amphoteric, containing both positive and negative charges and like all ampholytes exhibit the property that their charge depends on pH. At low pH (acidic conditions), proteins are positively charged while at high pH (basic conditions) they are negatively charged. For every protein there is a pH at which the protein is uncharged, the protein's isoelectric point. When a charged molecule is placed in an electric field it will migrate towards the opposite charge.

In a pH gradient such as those used in the present invention, containing a reducing agent such as dithiothreitol (DTT), a protein will migrate to the point at which it reaches its isoelectric point and becomes uncharged. The uncharged protein will not migrate further and stops. Each protein will stop at its isoelectric point and the proteins can thus be separated according to their isoelectric points. In order to achieve optimal separation of proteins, various pH gradients may be used. For example, a very broad range of pH, from about 3 to 11 or 3 to 10 can be used, or a more narrow range, such as from pH 4 to 7 or 5 to 8 or 7 to 10 or 6 to 11 can be used. The choice of pH range is determined empirically and such determinations are within the skill of the ordinary practitioner and can be accomplished without undue experimentation.

In the second dimension, proteins are separated according to molecular weight by measuring mobility through a uniform or gradient polyacrylamide gel in the detergent sodium dodecyl sulfate (SDS). In the presence of SDS and a reducing agent such as dithiothreitol (DTT), the proteins act as though they are of uniform shape with the same charge to mass ratio. When the proteins are placed in an electric field, they migrate into and through the gel from one edge to the other. As the proteins migrate though the gel, individual proteins move at different speeds with the smaller ones moving faster than the larger ones. This process is stopped when the fastest moving components reach the other side of the gel. At this point, the proteins are distributed across the gel with the higher molecular weight proteins near the origin and the low molecular weight proteins near the other side of the gel.

It is well known in the art that various concentration gradients of acrylamide may be used for such protein separations. For example, a gradient of from about 5% to 20% may be used in certain embodiments or any other gradient that achieves a satisfactory separation of proteins in the sample may be used. Other gradients would include but not be limited to from about 5 to 18%, 6 to 20%, 8 to 20%, 8 to 18%, 8 to 16%, 10 to 16%, or any range as determined by one of skill.

The end result of the 2D gel procedure is the separation of a complex mixture of proteins into a two dimensional array, a pattern of protein spots, based on the differences in their individual characteristics of isoelectric point and molecular weight.

### Reagents

Protease inhibitor cocktail were from Roche Diagnostics Corporation (Indianapolis, Ind.), Protein assay and purification reagents were from Bio-Rad Laboratories (Hercules, Calif.). Immobilon-P membranes and ECL reagents were from Pierce (Rockford, Ill.). All other chemicals were from Sigma Chemical (St. Louis, Mo.).

### 2D Gel Standards

Purified proteins having known characteristics are used as internal and external standards and as a calibrator for 2D gel electrophoresis. The standards consist of seven reduced, denatured proteins that can be run either as spiked internal standards or as external standards to test the ampholyte mixture and the reproducibility of the gels. A set mixture of proteins (the “standard mixture”) is used to determine pH gradients and molecular weights for the two dimensions of the electrophoresis operation. Table II lists the isoelectric point (pI) values and molecular weights for the proteins included in a standard mixture.

TABLE II

| Protein | pI | Molecular Weight (Da) |
|---|---|---|
| Hen egg white conalbumin | 6.0, 6.3, 6.6 | 76,000 |
| Bovine serum albumin | 5.4, 5.5, 5.6 | 66,200 |
| Bovine muscle actin | 5.0, 5.1 | 43,000 |
| Rabbit muscle GAPDH | 8.3, 8.5 | 36,000 |
| Bovine carbonic anhydrase | 5.9, 6.0 | 31,000 |
| Soybean trypsin inhibitor | 4.5 | 21,500 |
| Equine myoglobin conalbumin | 7.0 | 17,500 |

In addition, standard mixtures such as Precision Plus Protein Standards (Bio-Rad Laboratories), a mixture of 10 recombinant proteins ranging from 10-250 kD, are typically added as external molecular weight standards for the second dimension, or the SDS-PAGE portion of the system. The Precision Plus Protein Standards have an r² value of the Rf vs. log molecular weight plot of >0.99.

### Separation of Proteins in Serum Samples

An appropriate amount of isoelectric focusing (IEF) loading buffer (LB-2), was added to the diluted serum sample, incubated at room temperature and vortexed periodically until the pellet was dissolved to visual clarity. The samples were centrifuged briefly before a protein assay was performed on the sample.

Approximately 100 μg of the serum proteins were suspended in a total volume of 184 μl of IEF loading buffer containing 5 M urea, 2 M Thiourea, 1% CHAPS, 2% ASB-14, 0.25% Tween 20, 100 mM DTT, 1% ampholytes pH 3-10, 5% glycerol, 1×EDTA-free protease inhibitor cocktail and 1 μl Bromophenol Blue as a color marker to monitor the process of gel electrophoresis. Each sample was loaded onto an 11 cm IEF strip (Bio-Rad Laboratories), pH 5-8, and overlaid with 1.5-3.0 ml of mineral oil to minimize the sample buffer evaporation. Using the PROTEAN® IEF Cell, an active rehydration was performed at 50V and 20° C. for 12-18 hours.

IEF strips were then transferred to a new tray and focused for 20 min at 250V followed by a linear voltage increase to 8000V over 2.5 hours. A final rapid focusing was performed at 8000V until 20,000 volt-hours were achieved. Running the IEF strip at 500V until the strips were removed finished the isoelectric focusing process. Isoelectric focused strips were incubated on an orbital shaker for 15 min with equilibration buffer (2.5 ml buffer/strip). The equilibration buffer contained 6M urea, 2% SDS, 0.375M HC1, and 20% glycerol, as well as freshly added DTT to a final concentration of 30 mg/ml. An additional 15 min incubation of the IEF strips in the equilibration buffer was performed as before, except freshly added iodoacetamide (C₂H₄INO) was added to a final concentration of 40 mg/ml. The IPG strips were then removed from the tray using clean forceps and washed five times in a graduated cylinder containing the Bio Rad Laboratories running buffer 1× Tris-Glycine-SDS.

The washed IEF strips were then laid on the surface of Bio Rad pre-cast CRITERION SDS-gels 8-16%. The IEF strips were fixed in place on the gels by applying a low melting agarose. A second dimensional separation was applied at 200V for about one hour. After running, the gels were carefully removed and placed in a clean tray and washed twice for 20 minutes in 100 ml of pre-staining solution containing 10% methanol and 7% acetic acid.

### Staining and Analysis of the 2D Gels

Once the 2D gel patterns of the serum samples were obtained, the protein spots resolved in the gels were visualized with either a fluorescent or colored stain. In the preferred embodiment, the fluorescent dye SyproRuby™ (Bio-Rad Laboratories) was the stain. Once the protein spots had been stained, the gel was scanned by a digital fluorescent scanner or when visible dyes are employed, a digital visible light scanner, and a digital image of the protein spot pattern of the gel, i.e. a protein expression profile of the sample, was obtained.

The digital image of the scanned gel was processed using PDQuest™ (Bio-Rad Laboratories) image analysis software to first detect the proteins, locate the selected biomarkers, and then to quantitate the protein in each of the selected spots. The scanned image was cropped and filtered to eliminate artifacts using the image editing control. Individual cropped and filtered images were then placed in a matched set for comparison to other images and controls.

This process allowed quantitative and qualitative spot comparisons across gels and the determination of protein biomarker molecular weight and isoelectric point values. Multiple gel images were normalized to allow an accurate and reproducible comparison of spot quantities across two or more gels. The gels were normalized using the “total of all valid (detected and confirmed by the operator) spots method” in that a small percentage of the 1200 protein spots detected and verified change between serum samples, and that all spots detected and verified is a good estimate to correct for any differences in total protein amount applied to each gel. The quantitative amounts of the selected biomarkers present in each sample were then exported for further analysis using statistical programs.

### Tryptic Digestion, MALDI/MS, and LC-MS/MS

Following software analysis, unique spots were excised from the gel using the ProteomeWorks™ robotic spot cutter (Bio-Rad). In-gel spots were subjected to proteolytic digestion on a ProGest™ (Genomic Solutions, Ann Arbor, Mich.). A portion of the resulting digest supernatant was used for MALDI/MS analysis. Peptide solutions were concentrated and desalted using μ-C18 ZipTips™ (Millipore). Peptides were eluted with MALDI matrix alpha-cyano 4-hydroxycinnamic acid prepared in 60% acetonitrile, 0.2% TFA. Samples were robotically spotted onto MALDI chip, using ProMS™ (Genomic Solutions, Ann Arbor, Mich.).

MALDI/MS data was acquired on an Applied Biosystems Voyger DE-STR instrument and the observed m/z values were submitted to ProFound (Proteometrics software package) for peptide mass fingerprint searching using NCBInr database.

For LC/MS/MS, samples were analyzed by nano-LC/MS/MS on a Micromass Q-TOF 2. Aliquots of 15 μl of hydrolysate were processed on a 75 mm C18 column at a flow rate of 200 mL/min. MS/MS data were searched using a local copy of MASCOT, using peptide mass tolerance of ±100 ppm and fragment mass tolerance of ±0.1 Da, fixed modification of carbamidomethyl (C) and variables, including oxidation (M), acetyl (N-term), Pyro-glu (N-term Q), Pyro-glu (N-term E) and max missed cleavages of trypsin of 1.

### Biostatistical Analysis

Statistical significance of differences in biomarker blood serum concentrations between different patient and control groups is performed using methods well known in the art, Box and Whiskers plots and analysis of variance, employing a standard off the shelf software package, “Analyze-it” in Microsoft XL.

Discriminant analysis is a well-validated multivariate analysis procedure (27, 28). Discriminant analysis identifies sets of linearly independent functions that will successfully classify individuals into a well-defined collection of groups. The statistical model assumes a multivarate normal distribution for the set of biomarkers identified from each disease group. Let xᵢⱼ be the p-tuple vector of biomarkers from the i^{th} patient in the j^{th} group, j=1, 2 Let [symbol] be the p-tuple centroid of the j^{th} group, made up of the mean biomarker values from the j^{th} disease group. S is the estimate of the within group variance-covariance matrix. The discriminant function is then that set of linear functions determined by the vector a that maximizes the quantity:

$$\frac{{n}_{1} + {n}_{2}}{{n}_{1} {n}_{2}} \frac{{[ {\underline{a}}^{′} ( {\overline{x}}_{1} - {\overline{x}}_{2} ) ]}^{2}}{{\underline{a}}^{′} S \underline{a}}$$

The outcome of the discriminant analysis is a collection of m−1 linear functions of the biomarkers (m) that maximize the ability to separate individuals into disease groups. The vector a is the p-tuple vector which contains the coefficients that, when multiplied by an individual's biomarkers, produces the linear discriminant function, or index that is used to classify that individual.

In general, if there are m biomarkers, there will be a maximum of (m−1, g−1) discriminant functions where g is the number of groups. Let. aⱼ(k) be the k^{th} p-tuple discriminant function. Then the value of that discriminator for the i^{th} patient is aⱼ(k)′xᵢ. Thus for each patient there are k such values computed, which are used in a classification analysis. The discriminant functions themselves are linearly independent, i.e., for each pair of the m discriminant functions, aⱼ(k) and aⱼ(l), then aⱼ(k)′aⱼ(l)=0. Thus, the m−1 discriminant functions provide incremental and non-redundant discriminant ability.

Identifying the discriminant function involves identifying the coefficients λ from the linear algebraic system of equations |H−λᵢ(H+E)|=0 where H and E are the one way analysis of variance hypotheses and error matrices respectively. It is this computation that is provided by SAS. SAS identifies the collection of best discriminators using a forward entry procedure where the p-value to enter and the p value to stay in the model are each 0.15.

While the discrimination procedure is fairly robust in the presence of mild departures from the normality assumption, it is very sensitive to the assumption of homogeneity of variance. This means that the variance-covariance matrices of the groups between which discrimination is sought must be equal. In this circumstance, these variance-covariance matrices can be pooled. However, in the situation where the variance-covariance matrices are not equal (multivariate heteroscedasticity), this pooling procedure is sub-optimal. In this circumstance, the individual variance-covariance matrices are used.

The use of the two within-group variance-covariance matrices is an important complication in the computation of discriminant functions. When the homoscedasticity assumption is appropriate, the within group variance-covariance matrices can be pooled, producing a linear discriminant function. The use of the within-group variance-covariance matrices produces a quadratic discriminant function, (i.e., where the discriminant function is a function of the squares of the proteomic measures). Both linear and quadratic statistical functions are illustrated in the embodiments of this invention.

### Classification Analysis

Discriminant analysis was applied to the training set, from which the contribution of each individual biomarker was determined. The SAS® statistical software program was then used to determine the linear combinations of biomarkers that provided an optimum classification of individuals into disease groups. Alternatively, the programmer manually selected different combinations of biomarkers to be incorporated into a linear or quadratic discriminant function to optimize the classification of individuals into disease groups.

The output of discriminant analysis (DA) is a classification table that permits the calculation of clinical sensitivity, specificity, positive predictive value (PPV) and negative predictive value (NPV):
Clinical Sensitivity is how often the test is positive in diseased patients.
Clinical Specificity is how often the test is negative in non-diseased individuals.
Negative Predictive Value (NPV) is the probability that the patient will not have the disease when restricted to all individuals who test negative.
Positive Predictive Value (PPV) is the probability that the patient has the disease when restricted to those individuals who test positive.

NPV and PPV were not assessed in the case of the present study as these values are dependent upon patient mix and the present study used different numbers of patients in each category, due to sample availability.

### 2D Gel Electrophoretic Controls

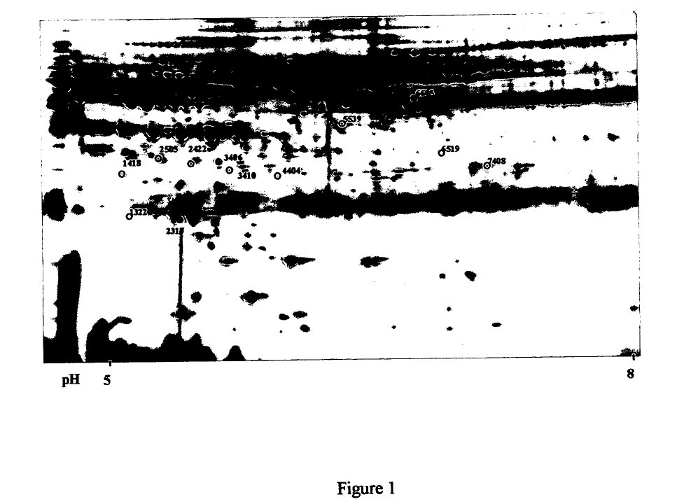

Representative samples from individuals with known cases of breast cancer, benign breast disease, or normal controls, were run as positive and negative reference controls. Serum containing all of the selected biomarkers was also provided as a reference standard. A reference control was periodically run as an external standard and for tracking overall performance and reproducibility. In addition, 2D gel images from samples classified as breast cancer, benign breast disease, or normal controls, were used for reference. The spot locations for the selected biomarkers were illustrated in FIG. 1.

### Samples Analyzed

The present invention is a two-dimensional gel electrophoresis assay of patient blood serum samples, employing the 12 biomarker spots, combined with multivariate biostatistics, is used to distinguish between subjects with normal breasts, patients with benign breast disease, and patients with breast cancer.

The 2D gel electrophoresis of the human blood serum samples of this study separated >1200 spots in the pH 5-8 range, 12 of which (FIG. 1, numbered spots: 1322, 1418, 2317, 2422, 2525, 3406, 3410, 4404, 5539, 6505, 6519, and 7408) displayed differences in serum concentrations between samples from normal subjects, patients with benign breast disease or abnormalities, and patients with breast cancer, as well as breast cancer.

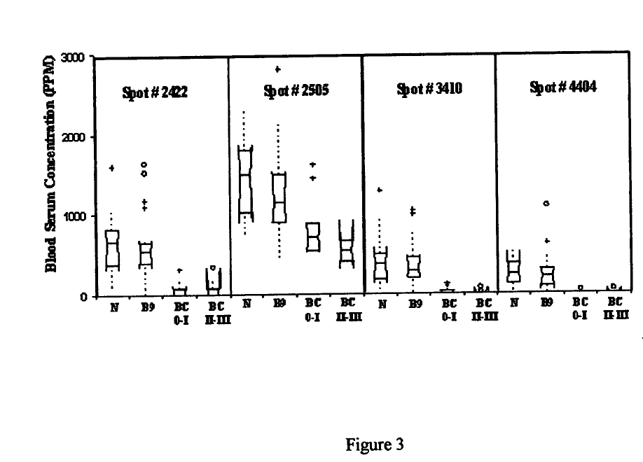

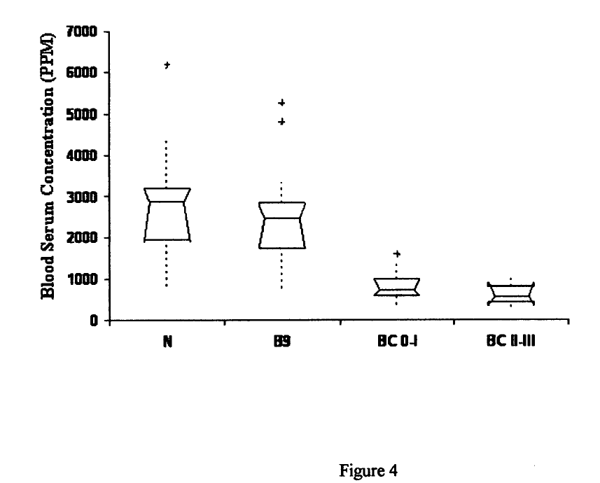

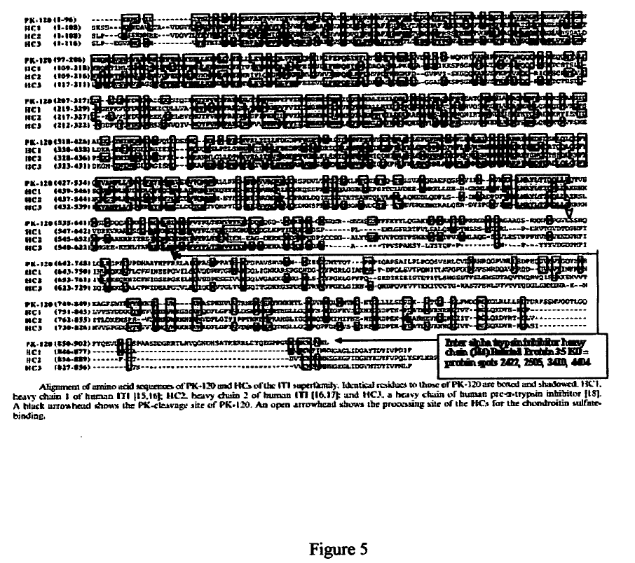

Biomarker protein spots 2422, 2505, 3410, and 4404, correspond to electrophoretic variants of the 35 KD processing product of inter-alpha-trypsin inhibitor heavy chain (H4) related protein (table XI), isoforms 1 and 2 (FIGS. 1, 5, table XII, XIII). These four spots separately (FIG. 3) and collectively (FIG. 4) demonstrate progressive down-shifts in blood serum concentration, with statistically significant single variable biostatistics (table IV).

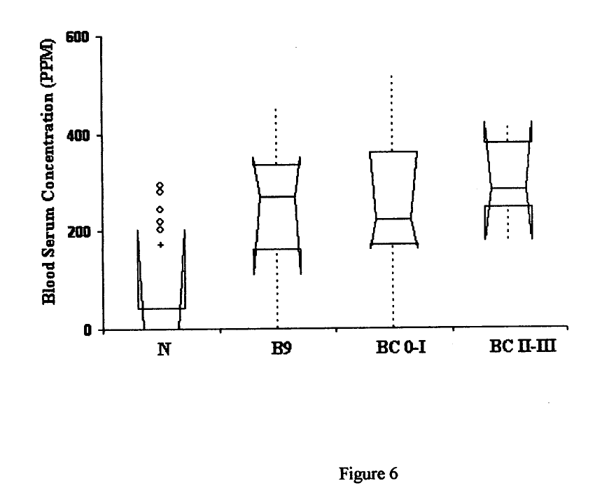

As shown in FIG. 6, biomarker protein spot #1322, Immunoglobulin lambda (λ) light chain (table XXII) demonstrates an early and pronounced up shift in blood serum concentration, in the transition between normal (N) and benign (B9), with that higher concentration maintained through the earlier (BC0-I) and later stages (BCII-III) of breast cancer. The statistical significance of this early rise (table V) demonstrates the potential for early detection, where 72.7% of the normal subjects ( 16/22) have no detectable (0 values) concentration of the marker in their sera compared to only 3.4% ( 2/58) of the B9-BCIII patients have 0 values in their sera.

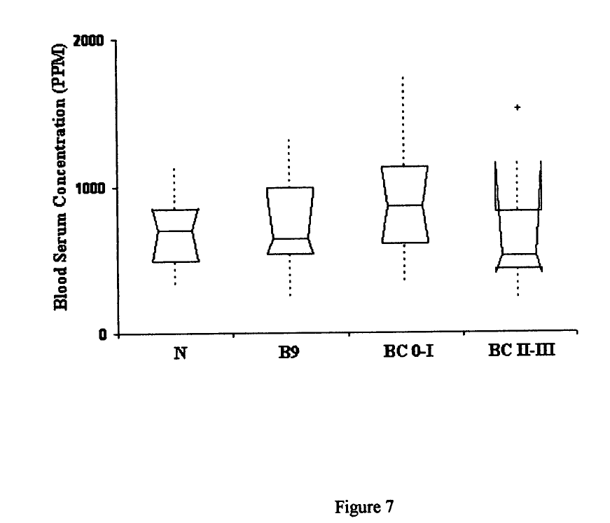

As shown in FIG. 7, biomarker protein spot #1418, alpha-1-microglobulin (table XVII) demonstrates a statistically significant rise in blood serum concentrations only in the earlier stages of breast cancer (BC0-I), compared to normal control subjects (table VI).

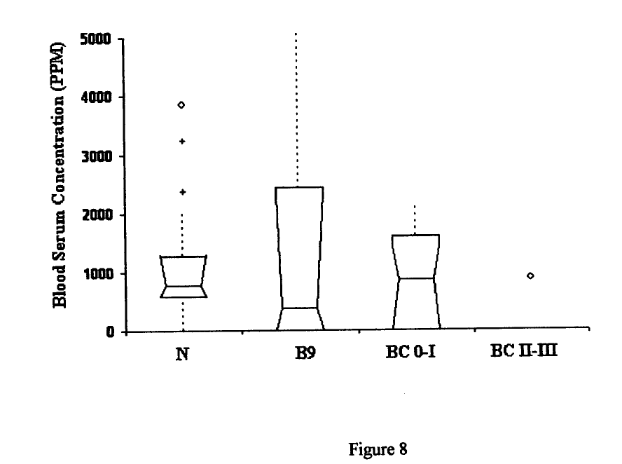

As shown in FIG. 8, biomarker protein spot #2317, Apolipoprotein A-I (Tables XVI), demonstrates a pronounced drop (table VII) in blood serum concentration in the later stage breast cancer (BC II-III), where this biomarker is not detected (0 value) in blood serum from 4.5% ( 1/22) of the normal subjects (N), 37.5% ( 9/24) of the benign (B9) and 33.3% ( 8/24) of earlier stages of breast cancer (BC0-I), compared to the marker absence in 90.9% ( 10/11) in later stages breast cancer (BCII-III) patients. This indicates the capacity of biomarker protein spot 2317 for detection of more severe breast cancer.

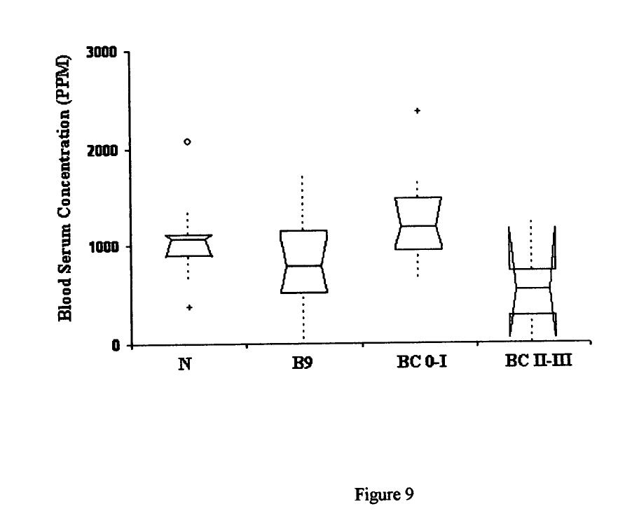

As shown in FIG. 9, biomarker protein spot #3406, Apolipoprotein E3 (Tables XV), there is a pronounced and statistically significant reduction in the blood serum concentration of this biomarker (Tables VIII) in later stage breast cancer (BC II-III) compared to normal control subjects and earlier stage breast cancer (BC 0-I) patients. It also indicates the significant high level of apolipoprotein E3 in the serum of BC 0-I compared to benign (B9) patients.

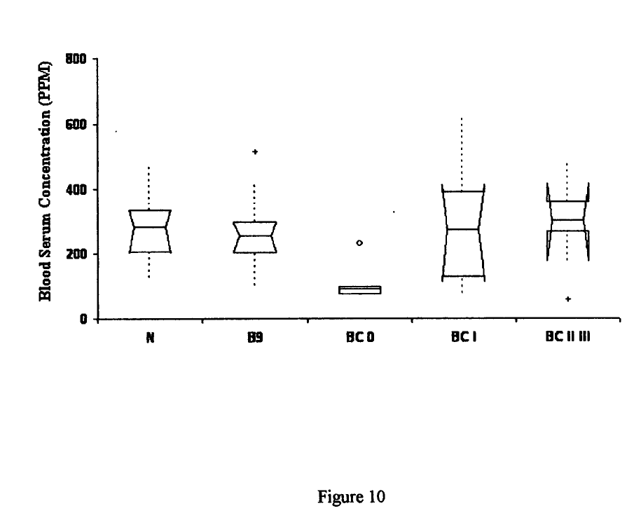

As shown in FIG. 10, biomarker protein spot #6519, Lectin P35 (Tables XIV), there is a statistically significant drop in blood serum concentration of biomarker protein spot 5539 (table IX), in earlier breast cancer (BC 0) patients compared to normal control subjects, benign (B9) patients and other breast cancer stages (BC I and BC II-III)

While individual single variable non-parametric statistics indicated no single biomarker was capable of fully distinguishing between normal samples, benign samples, and breast cancer samples, due to overlaps multivariate linear and quadratic discriminant analysis (Table X) indicated that the 12 biomarkers employed as a group were capable of discrimination of the three groups from each other (3-way, A & B) and between cancer and not cancer (2 way, C & D) with high sensitivities and specificities

When the 12 biomarker spots were robotically excised, subjected to in-gel trypsin digestion and the peptides analyzed by LC-MS/MS fingerprint identification, (Tables III), comparison of the 2D gel measured and the protein sequence calculated masses and isoelectric points of the biomarker spots, with the peptides identified by LC-MS/MS, indicated that some of the biomarker protein spots appear on 2D gels as smaller components of parent molecules, i.e. smaller than the original translation products of the mRNA, whereas others are the full length translated products, including those with additional molecular weight contribution from post-synthetic modifications, such as glycosylation, etc.

The serum samples may also be subjected to various other techniques known in the art for separating and quantitating proteins. Such techniques include, but are not limited to gel filtration chromatography, ion exchange chromatography, reverse phase chromatography, affinity chromatography (typically in an HPLC or FPLC apparatus), or any of the various centrifugation techniques well known in the art. Certain embodiments would also include a combination of one or more chromatography or centrifugation steps combined via electrospray or nanospray with mass spectrometry or tandem mass spectrometry of the proteins themselves, or of a total digest of the protein mixtures. Certain embodiments may also include surface enhanced laser desorption mass spectrometry or tandem mass spectrometry, or any protein separation technique that determines the pattern of proteins in the mixture either as a one-dimensional, two-dimensional, three-dimensional or multi-dimensional protein pattern, and or the pattern of protein post synthetic modification isoforms.

Quantitation of a protein by antibodies directed against that protein is well known in the field. The techniques and methodologies for the production of one or more antibodies to the proteins, routine in the field and are not described in detail herein.

As used herein, the term antibody is intended to refer broadly to any immunologic binding agent such as IgG, 1gM, IgA, IgD and IgE. Generally, IgG and/or 1gM are preferred because they are the most common antibodies in the physiological situation and because they are most easily made in a laboratory setting.

Monoclonal antibodies (MAbs) are recognized to have certain advantages, e.g., reproducibility and large-scale production, and their use is generally preferred. The invention thus provides monoclonal antibodies of human, murine, monkey, rat, hamster, rabbit and even chicken origin. Due to the ease of preparation and ready availability of reagents, murine monoclonal antibodies are generally preferred. However, “humanized” antibodies are also contemplated, as are chimeric antibodies from mouse, rat, or other species, bearing human constant and/or variable region domains, bispecific antibodies, recombinant and engineered antibodies and fragments thereof.

The term “antibody” thus also refers to any antibody-like molecule that has 20 an antigen binding region, and includes antibody fragments such as Fab′, Fab, F(ab′)2, single domain antibodies (DABS), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Antibodies to the one or more of the 12 protein biomarkers may be used in a variety of assays in order to quantitate the protein in serum samples, or other fluid or tissue samples. Well known methods include immunoprecipitation, antibody sandwich assays, ELISA and affinity chromatography methods that include antibodies bound to a solid support. Such methods also include microarrays of antibodies or proteins contained on a glass slide or a silicon chip, for example.

It is contemplated that arrays of antibodies to up to 12 protein biomarkers, or peptides derived, may be produced in an array and contacted with the serum samples or protein fractions of serum samples in order to quantitate the proteins. The use of such microarrays is well known in the art and is described, for example in U.S. Pat. No. 5,143,854, incorporated herein by reference.

The present invention includes a screening assay for breast cancer based on the up-regulation and/or down-regulation of the 12 protein biomarkers. One embodiment of the assay will be constructed with antibodies recognizing up to 12 protein biomarkers. One or more antibodies targeted to antigenic determinants of up to 12 protein biomarkers will be spotted onto a surface, such as a polyvinyl membrane or glass slide. As the antibodies used will each recognize an antigenic determinant of up to 12 protein biomarkers, incubation of the spots with patient samples will permit attachment of up to 12 protein biomarkers to the antibody.

The binding of up to 12 protein biomarkers can be reported using any of the known reporter techniques including radioimmunoassays (RIA), stains, enzyme linked immunosorbant assays (ELISA), sandwich ELISAs with a horseradish peroxidase (HRP)-conjugated second antibody also recognizing up to 12 protein biomarkers, the pre-binding of fluorescent dyes to the proteins in the sample, or biotinylating the proteins in the sample and using an HRP-bound streptavidin reporter. The HRP can be developed with a chemiluminescent, fluorescent, or colorimetric reporter. Other enzymes, such as luciferase or glucose oxidase, or any enzyme that can be used to develop light or color can be utilized at this step.

As shown in FIG. 5, the N-terminal of the 3 isoforms of ITIHRP (HC1, HC2, HC3) shows substantial homology with isoform of heavy chain 4 (Pk-120). However, the sequence containing the 35 KD (PK-120), corresponds to biomarkers 2422, 2595, 3410, and 4404 of the present invention show a substantially decreased homology in the C-terminal sequence and the lack of homology is maintained throughout the 35 KD product. For high throughput immunoassays, biomarker specific antibodies can be developed using truncated cDNA sequences to produce recombinant antigens in bacterial or mammalian systems, containing only the epitopes of the 35 KD biomarkers without the epitopes of the upstream region of the parent molecules. These antigens in turn can be used to immunize rabbits, sheep, chickens, or goats, for polyclonal antibodies, or mice to produce monoclonal antibodies either with classic hybridoma technologies or phage display methods. The recombinant antigens can also be employed as affinity agents to purify antibodies and as reagent controls in assays.

Alternatively, antibodies could be raised to the upstream portions of the parent molecule that would cross react with the ITIH 1-3 species (HC-1, HC-2, HC-3, FIG. 5) as well, due to the substantial homology in these regions. Such antibodies could be used as affinity capture agents to isolate from serum or other sources the family of ITIHs, i.e. ITIH 1-3 and ITIHRP. Subsequent treatment of this group with plasma Kallikrein, which selectively cleaves out the ITIHRP would release the 35 KD ITHIL species which would not bind the antibodies and thus the biomarkers, in native purified form, can be obtained from a biological sample.

Similar approaches are available for the other biomarkers whose amino acid sequences are defined in some of the accompanying tables.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention.

More specifically, it is well recognized in the art that the statistical data, including but not limited to the mean, standard error, standard deviation, median, interquartile range, 95% confidence limits, results of analysis of variance, non-parametric median tests, discriminant analysis, etc., will vary as data from additional patients are added to the database or antibodies are utilized to determine concentrations of one or more of the 12 biomarkers of the present invention, or any biomarker. Therefore changes in the statistical values of one or more of the 12 protein biomarkers do not depart from the concept, spirit and scope of the invention.

Also more specifically, it is disclosed (in cross referenced US Utility Patent Applications by Goldknopf, I. L., et al. Ser. Nos. 11/507,337 and 11/503,881, US Provisional Patent Applications by Goldknopf et al. Ser. No. 60/708,992 and 60/738,710, and referenced in Goldknopf, I. L et al. 2006 and Sheta et al. 2006, hereby incorporated as reference) that blood serum concentrations of protein biomarkers, including an inter alpha trypsin inhibitor family heavy chain (H4) related protein 35 KD and Apolipoprotein E3, can be used in combination with other biomarkers for diagnosis, differential diagnosis, and screening. Consequently, the use of one or more of the 12 protein biomarkers in conjunction with one or more additional biomarkers not disclosed in the present invention does not depart from the concept, spirit and scope of the invention.

It is also well recognized in the art that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

TABLE III
Biomarker Protein Identification by LC MSMS of
2D gel spot in-gel trypsin digests.

| | | | # of |
|---|---|---|---|
| Biomarker | | Accession | Peptides |
| Spot # | Protein ID | # | Matched |
| 1332 | Immunoglobuline lambda chain | 106653 | 2 |
| 1418 | Alpha-1-microglobulin | 223373 | 3 |
| 2317 | Proapolipoprotein | 178775 | 9 |
| 2422 | Inter-α-trypsin inhibitor family heavy | 1483187 | 5 |
| | chain related protein (ITIHRP) | | |
| 2505 | Inter-α-trypsin inhibitor family heavy | 1483187 | 3 |
| | chain related protein (ITIHRP) | | |
| 3406 | Apolipoprotein E3 | 178849 | 3 |
| | | 1942471 | 4 |
| 3410 | Inter-α-trypsin inhibitor family heavy | 1483187 | 4 |
| | chain related protein (ITIHRP) | | |
| 4404 | Inter-α-trypsin inhibitor family heavy | 1402590 | 3 |
| | chain related protein (ITIHRP) | | |
| 5539 | Serum Albumin | 28590 | 5 |
| 6519 | Lectin P35 | 1669349 | 3 |
| 6605 | Transferrin | 4557871 | 9 |
| 7408 | Complement component C4A | 179674 | 2 |

TABLE IV
Mean Serum level (PPM) of the 4 isoforms of ITIHRP
(spots # 2422, 2505, 3410 and 4404)

| Spot | classification (# of subjects) | | | |
|---|---|---|---|---|
| Num- | N | B9 | BC 0-I | BC II-III |
| ber | (22) | (24) | (24) | (11) |
| 2422 | 619.9 ± 75.7 | 646.0 ± 83.2 | 46.6 ± 16.2* | 62.8 ± 32.9* |
| 2505 | 1542.0 ± 140.7 | 1301.6 ± 106.3 | 780.8 ± 67.5* | 546.4 ± 54.8* |
| 3410 | 424.8 ± 64.7 | 405.6 ± 54.9 | 17.1 ± 6.5* | 7.8 ± 6.5* |
| 4404 | 290.6 ± 37.5 | 279.5 ± 50.4 | 1.4 ± 1.4* | 3.8 ± 3.8* |

*Significantly different from Normal Control (N) and from Benign (B9) (P < 0.0001)

TABLE X
Application of Quadratic (A, C) and Linear (B, D) Discriminant Analysis of
the blood serum concentrations of the 12 biomarkers to diagnosis and differential
diagnosis of normal individuals (N), patients with benign breast disease or abnormalities
(B9), and patients with breast cancer (BC); as well as for screening of patients with
cancer (Cancer) and/or patients without cancer (Not Cancer).

| | Quadratic Discriminant Analysis-3 | | | | | | Linear Discriminant Analysis-3 | | | | |
|---|---|---|---|---|---|---|---|---|---|---|---|
| | Way = N vs. B9 vs. BC | | | | | | Way = N vs. B9 vs. BC | | | | |
| | | Classified As | | | | | | Classified As | | | |
| | From Diagnosis | N | B9 | BC | Total | | From Diagnosis | N | B9 | BC | Total |
| A | N | 21 | 0 | 0 | 21 | B | N | 18 | 2 | 1 | 21 |
| | | (100%) | (0%) | (0%) | (100%) | | | (86%) | (10%) | (5%) | (100%) |
| | B9 | 3 | 32 | 3 | 38 | | B9 | 10 | 25 | 3 | 38 |
| | | (8%) | (84%) | (8%) | (100%) | | | (26%) | (66%) | (8%) | (100%) |
| | BC | 0 | 1 | 38 | 39 | | BC | 2 | 3 | 34 | 39 |
| | | (0%) | (3%) | (97%) | (100%) | | | (5%) | (8%) | (87%) | (100%) |
| | Total | 24 | 33 | 41 | 98 | | Total | 30 | 30 | 38 | 98 |
| | | (24%) | (34%) | (42%) | (100%) | | | (31%) | (31%) | (39%) | (100%) |

| | Quadratic Discriminant | | | | | Linear Discriminant Analysis-2 | | | |
|---|---|---|---|---|---|---|---|---|---|
| | Analysis-2 Way = N-B9 vs. BC | | | | | Way = N-B9 vs. BC | | | |
| | | Classified As | | | | | Classified As | | |
| | From Diagnosis | Not Cancer | Cancer | Total | | From Diagnosis | Not Cancer | Cancer | Total |
| C | Not Cancer | 56 | 3 | 59 | D | Not Cancer | 55 | 4 | 59 |
| | | (95%) | (5%) | (100%) | | | (93%) | (7%) | (100%) |
| | Cancer | 1 | 38 | 39 | | Cancer | 3 | 34 | 39 |
| | | (3%) | (97%) | (100%) | | | (8%) | (87%) | (100%) |
| | Total | 57 | 41 | 98 | | Total | 58 | 38 | 98 |
| | | (58%) | (42%) | (100%) | | | (59%) | (39%) | (100%) |

TABLE XI
Amino acid sequence of Inter-alpha-Trypsin inhibitor heavy chain (H4) related protein (ITIHRP):
Spots # 2422, 2505, 3410, 4404

Protein Alternative Names:
IHRP; ITIHL1, 2; PK120
INTER-ALPHA-TRYPSIN INHIBITOR, HEAVY CHAIN 4
INTER-ALPHA-TRYPSIN INHIBITOR, HEAVY CHAIN-LIKE, 1, 2
INTER-ALPHA-TRYPSIN INHIBITOR, HEAVY CHAIN-RELATED PROTEIN
PLASMA KALLIKREIN-SENSITIVE GLYCOPROTEIN 120
Inter-alpha (globulin) inhibitor H4 (plasma Kallikrein-sensitive glycoprotein)
Parental Protein Full Sequence: NCBI accession # 1483187:
The tryptic peptide 35 kD processing product of ITIHRP is underlined

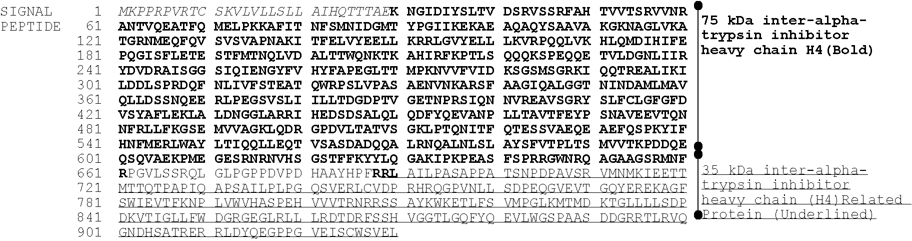

The amino acid sequence of the inter-aipha-trypsin inhibitor heavy chain (H4) related protein composed of 930 amino acids (Mwt 103.4 kDa). The N-terminal 28 residues corresponded to a signal peptide for secretion. The N-terminal 600 residues of the mature form exhibited considerable homology to those of inter-alpha trypsin inhibitor (ITI) heavy chains, while the C-terminal 300 residues showed no homology with the heavy chains and low homology with ATP-dependent proteases. Inter-alpha-trypsin inhibitor heavy chain (H4) related protein is readily cleaved into 75- and 35-kDa fragments when plasma is incubated at 37 degrees C. The cleaved site, Arg-Arg-Leu (RRL), is within a proline-rich region (Saguchi et al, J Biochem (1995) 117:14-18). The 35-kDa cleavage fragment (underlined), expands the amino acid sequence starting at Arginine (R)-689 to Leucine (L)-930, is the fragment detected on 2D gel electrophoresis, marked as spots # 2422, 2505, 3410, and 4404 (Mwt 35 KD), it is most likely that the 4 protein spots corresponds to the 35 KD processing product in depicted in FIG. 1. [00510050] The sequence of peptides also exists in proteins with NCBI accession numbers: 1483187; 4096840; 7770149; 13432192; 55620443; 55732844, which belong to “Inter-alpha-trypsin inhibitor family heavy chain (H4) related protein family (ITIHRP; ITIH4).

TABLE XII
Amino acid sequence of inter alpha trypsin inhibitor heavy chain (H4) related
protein 35 KD processing products of Isoforms 1 and 2
LC/MS/MS identified peptides span (underlined):

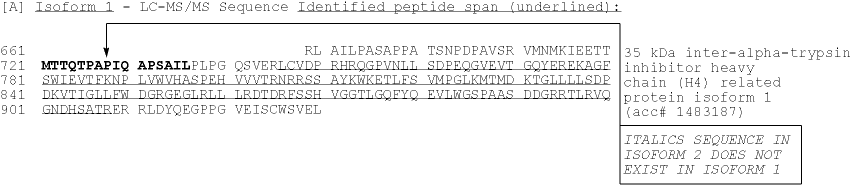

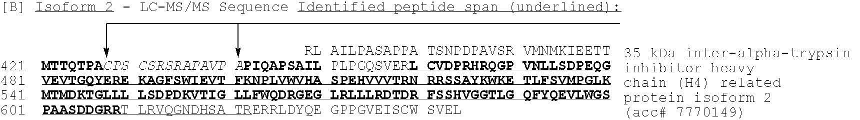

TABLE XIII
Sequence alignment of ITIHRP isoforms 1 and 2

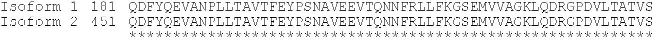

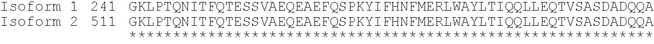

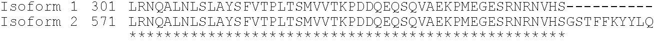

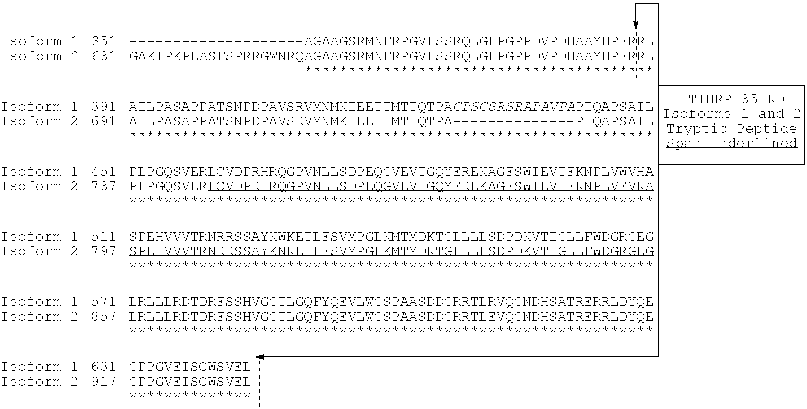

TABLE XIV
Amino acid sequence of human Lectin P35 (Spot #6519)

Protein alternative names:

Ficolin-2 precursor (Collagen/fibrinogen domain-containing
protein 2) (Ficolin-B) (Ficolin B) (Serum Lectin p35)
(EBP-37) (Heckling) (L-Ficolin).
Parental Protein Full Sequence: NCBI accession #1669349:
LC/MS/MS identified peptides span underlined:

| | |
|---|---|
| 1 | MELDRAVGVL GAATLLLSFL GMAWALQAAD TCPEVKMVGL EGSDKLTILR GCPGLPGAPG |
| 61 | DKGEAGTNGK RGERGPPGPP GKAGPPGPNG APGEPQPCLT GPRTCKDLLD RGHFLSGWHT |
| 121 | IYLPDCRPLT VLCDMDTDGG GWTVFQRRVD GSVDFYRDWA TYKQGFGSRL GEFWLGNDNI |
| 182 | HALTAQGTSE LRVDLVDFED NYQFAKYRSF KVADEAEKYN LVLGAFVEGS AGDSLTFHNN |
| 241 | QSFSTKDQDN DLNTGNCAVM FQGAWWYKNC HVSNLNGRYL RGTHGSFANG INWKSGKGYN |
| 301 | YSYKVSEMKV RPA |

TABLE XV
Amino acid sequence of Apolipoprotein E3 (spot # 3406)

Protein alternative names:
AD2; BROAD-BETALIPOPROTEINEMIA; FLOATING-BETALIPOPROTEINEMIA; MGC1571;
apoprotein
APOE APOLIPOPROTEIN E, DEFICIENCY OR DEFECT OF
Alzheimer disease 2 (APOE*E4-associated, late onset)
CORONARY ARTERY DISEASE, SEVERE, SUSCEPTIBILITY TO
DYSBETALIPOPROTEINEMIA DUE TO DEFECT IN APOLIPOPROTEIN E-d
FAMILIAL HYPERBETA- AND PREBETALIPOPROTEINEMIA
FAMILIAL HYPERCHOLESTEROLEMIA WITH HYPERLIPEMIA
HYPERLIPEMIA WITH FAMILIAL HYPERCHOLESTEROLEMIC XANTHOMATOSIS
HYPERLIPOPROTEINEMIA, TYPE III
Apolipoprotein E
Apolipoprotein E precursor
Apolipoprotein E3
Parental Protein Full Sequence:
NCBI accession # 1669349 and accession # 178849:
LC/MS/MS identified peptides span underlined:

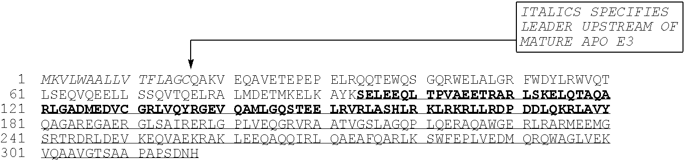

TABLE XVI
Amino acid sequence of Apolipoprotein A-I (spot #2317)

Protein alternative names:

Amyloidosis
APOLIPOPROTEIN OF HIGH DENSITY LIPOPROTEIN APOA1/APOC3 FUSION GENE
Apolipoprotein A-I
Apolipoprotein A-I precursor
Proapolipoprotein
Parental Protein Full Sequence: NCBI accession #178775:
LC/MS/MS identified peptides span underlined:

| | | |
|---|---|---|
| 1 | RHFWQQDEPP QSPWDRVKDL ATVYVDVLKD SGRDYVSQFE GSALGKQLNL KLLDNWDSVT | SEQUENCE IDENTICAL TO |
| 61 | STFSKLREQL GPVTQEFWDN LEKETEGLRQ EMSFWLEEVK AKVQPYLDDF QKKWQEEMEL | APOLIPOPROTEIN A1 LACKING |
| 121 | YRQKVEPLRA ELQEGARQKL HELQEKLSPL GEEMRDRARA HVDALRTHLA PYSDELRQRL | THE N-TERMINAL, SIGNAL |
| 181 | AARLEALKEN GGARLAEYHA KATEHLSTLS EKAKPALEDL RQGLLPVLES FKVSFLSALE | PEPTIDE [MKAAVLTLAVLFLTGSQA] |
| 241 | EYTKKLNTQ | |

TABLE XVII
Amino acid sequence of Alpha-1-microglobulin (spot #1418)

Protein alternative names:

HCP; IATIL; ITIL; OTTHUMP00000063975; UTI
ALPHA-1-MICROGLOBULIN/BIKUNIN PRECURSOR
Alpha-1-microglobulin/bikunin precursor (inter-alpha-trypsin inhibitor,
light chain; protein HG)
Alpha-1-microglobulin/bikunin precursor; inter-alpha-trypsin
COMPLEX-FORMING GLYCOPROTEIN HETEROGENEOUS IN CHARGE INTER-ALPHA-TRYPSIN
INHIBITOR
Amino acid secjuence: NCBI accession #223373: Alpha-1-microglobulin
LC/MS/MS identified peptides span underlined:

| | |
|---|---|
| 1 | GPVPTPPDNI QVQENFNISR IYGKWYNLAI GSTCPLKIMD RMTVSTLVLG EGATEAEISM |
| 61 | TSTRWRKGVC EETSGAYEKT DTDGKFLYHK SKWNITMESY VVHTNYDEYA IFLTKKFSRH |
| 121 | HGPTITAKLY GRAPQLRETL LQDFRVVAQG VGIPEDSIFT MADRGECVPG EQEPEPILIP |
| 181 | R |

The alpha-1-microglobulin (Protein HC) is a 31-kD, single chain plasma glycoprotein, which appears
to be involved in regulation of the inflammatory process (Mendez et al., 1986). The alpha-1-
microglobulin/bikunin precursor gene (AMBP) codes for a precursor that splits into alpha-1-
microglobulin, which belongs to the lipocalin superfamily, and bikunin (formerly HI-30, urinary
trypsin inhibitor, inhibitor subunit of inter-alpha-trypsin inhibitor). The amino acid sequence of
he parental protein is provided below:
Parental Protein alternative names:
Alpha-1-microglobulin (Protein HC) (Complex-forming glycoprotein heterogeneous in charge)/Inter-
alpha-trypsin inhibitor light chain (ITI-LC) (Bikunin) (HI-30)]complex:
Parental protein sequence:

| | | | |
|---|---|---|---|
| Signal | MRSLGALLLL LSACLAVSAG PVPTPPDNIQ VQENFNISRI YGKWYNLAIG STCPWLKKIM | 60 | Alpha-I-microgpeptide |
| peptide | DRMTVSTLVL GEGATEAEIS MTSTRWRKGV CEETSGAYEK TDTDGKFLYH KSKWNITMES | 120 | |
| | YVVHTNYDEY AIFLTKKFSR HHGPTITAKL YGRAPQLRET LLQDFRVVAQ GVGIPEDSIF | 180 | |
| | TMADRGECVP CEQEPEPILI PRVRRAVLPQ EEEGSGGGQL VTEVTKKEDS CQLGYSAGPC | 240 | Inter-α-Trypsin Inhibitor- |
| | MGMTSRYFYN GTSMACETFQ YGGCMGNGNN FVTEKECLQT CRTVAACNLP IVRGPCRAFI | 300 | light chain (Bikunin) |
| | QLWAFDAVKG KCVLFPYGGC QGNGNKFYSE KECREYCGVP GDGDEELLRE SN | 352 | |

TABLE XVIII
Amino acid sequence of Complement C4A gamma (spot #7408)

Protein alternative names:

C4A2; C4A3; C4A4; C4A6; C4S; CO4
C4A anaphylatoxin
COMPLEMENT COMPONENT 4S
RODGERS FORM OF C4 COMPLEMENT COMPONENT 4A DEFICIENCY
acidic C4
c4 propeptide
complement component 4A preproprotein
complement component C4B
Amino acid sequence of C4A-gamma chain (spot #7408): Tryptic peptide span underlined
pI of Protein: 6.4
Protein MW: 33074 Da
1 11 21 31 41 51 61 71
EAPKVVEEQE SRVHYTVCIW RNGKVGLSGM AIADVTLLSG FHALRADLEK LTSLSDRYVS HFETEGPHVL LYFDSVPTSR
81 91 101 111 121 131 141 151
ECVGFEAVQE VPVGLVQPAS ATLYDYYNPE RRCSVFYGAP SKSRLLATLC SAEVCQCAEG KCPRQRRALE RGLQDEDGYR
161 171 181 191 201 211 221 231
MKFACYYPRV EYGFQVKVLR EDSRAAFRLF ETKITQVLHF TKDVKAAANQ MRNFLVRASC RLRLEPGKEY LIMGLDGATY
241 251 261 271 281 291
DLEGHPQYLL DSNSWTEEMP SERLCRSTRQ RAACAQLNDF LQEYGTQGCQ V

TABLE XIX
Amino acid sequence of parental protein Complement C4A: NCBI Accession # 179674

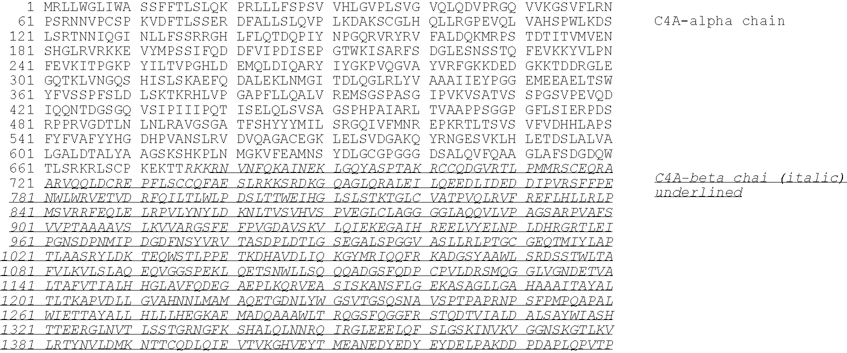

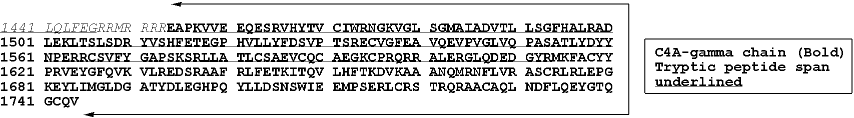

TABLE XX
Amino acid sequence of Transferrin (spot #6605):

pI of the Protein: 6.8
Molecular Weight: 77050 Da
Protein Sequence: NCBI Accession #4557871

| | | |
|---|---|---|
| 1 | MRLAVGALLV CAVLGLCLAV PDKTVRWCAV SEHEATKCQS FRDHMKSVIP SDGPSVACVK | Peptides span |
| 61 | KASYLDCIRA IAANEADAVT LDAGLVYDAY LAPNNLKPVV AEFYGSKEDP QTFYYAVAVV | of spot #6606 |
| 121 | KKDSGFQMNQ LRGKKSCHTG LGRSAGWNIP IGLLYCDLPE PRKPLEKAVA NFFSGSCAPC | are underlined |
| 181 | ADGTDFPQLC QLCPGCGCST LNQYFCYSGA FKCLKDGAGD VAFVKHSTIF ENLANKADRD | |
| 241 | QYELLCLDNT RKPVDEYKDC HLAQVPSHTV VARSMQCKED LIWELLNQAQ EHFGKDKSKE | |
| 301 | FQLFSSPHGK DLLFKDSAHG FLKVPPRMDA KMYLGYEYVT AIRNLREGTC PEAPTDECKP | |
| 361 | VKWCALSHHE RLKCDEWSVN SVGKIECVSA ETTEDCIAKI MMGEADAMSL DGGFVYIAGK | |
| 421 | CGLVPVLAEN YNKSDNCEDT PEAGYFAVAV VKKSASDLTW DNLKGKKSCH TAVGRTAGWN | |
| 481 | IPMGLLYNKI NHCRFDEFFS ECCAPCSKKD SSLCKLCMGS GLNLCEPNNK ECYYCYTGAF | |
| 541 | RCLVEKGDVA FVKHQTVPQN TGGKNPDPWA KNLNEKDYEL LCLDGTRKPV EEYANCHLAR | |
| 601 | APNHAVVTRK DKEACVHKIL RQQQHLFCSN VTDCSGNFCL FRSETKDLLF RDDTVCLAKL | |
| 661 | HDRNTYEKYL GEEYVKAVGN LRKCSTSSLL EACTFRRP | |

TABLE XXI
Amino acid sequence of human albumin (spot #5539)

Protein alternative names:

DKFZp779N1935; PRO1341
ALB DYSALBUMINEMIC HYPERTHYROXINEMIA
HYPERTHYROXINEMIA, DYSALBUMINEMIC
PRO0883 protein
albumin precursor
serum albumin
Cell growth inhibiting protein 42
Protein sequence of Human Albumin, NCBI accession #28590:
|LC-MS/MS peptides span underlined.

| | |
|---|---|
| 1 | MKWVTFISLL FLFSSAYSRG VFRRDAHKSE VAHRFKDLGE ENFKALVLIA FAQYLQQCPF |
| 61 | EDHVKLVNEV TEFAKTCVAD ESAENCDKSL HTLFGDKLCT VATLRETYGE MADCCAKQEP |
| 121 | GRNECFLQHK DDNPNLPRLV RPEVDVMCTA FHDNEETFLK KYLYEIARRH PYFYAPELLF |
| 181 | FAKRYKAAFT ECCQAADKAA CLLPKLDELR *DEGKASSAKQ RLKCASLQKF GERAFKAWAV |
| 241 | ARLSQRFPKA EFAEVSKLVT DLTKVHTECC HGDLLECADD RADLAKYICE NQDSISSKLK |
| 301 | ECCEKPLLEK SHCIAEVEND EMPADLPSLA ADFVESKDVC KNYAEAKDVF LGMFLYEYAR |
| 361 | RHPDYSVVLL LRLAKTYETT LEKCCAAADP HECYAKVFDE FKPLVEEPQN LIKQNCELFE |
| 421 | QLGEYKFQNA LLVRYTKKVP EVSTPTLVEV SRNLCKVCSK CCKHPEAKRM PCAEDYLSVV |
| 481 | LNQLCVLHEK TPVSDRVTKC CTESLVNRRP CFSALEVDET YVPKEFNAET FTFHADICTL |
| 541 | SEKERQIKKQ TALVELVKHK PKATKEQLKA VMDDFAAFVE KCCKADDKET CFAEEGKKLV |
| 601 | AASQAALGL |

*Protein sequence that corresponds to spot #5539 has an estimated molecular weight of ~45 kD and pI of ~6.2, which is calculated to correspond to albumin fragment sequence that starts at Aspartic acid (D) residue number 211* extends to the C-terminal Leucine (L) residue #609 and expands the LC-MS/MS identified peptides (underlined).

TABLE XXII

Amino acid sequence of Immunoglobulin lambda chain (spot #1322):
NCBI accession #106653 peptide span underlined:

| | |
|---|---|
| 1 | MAWTVLLLGL LSHCTGSVTS YVLTQPPSVS VAPGKTASIT CGGNNIGSKS VHWYQQKPGQ |
| 61 | APVLVVYDDS DRPSGIPERF SGSNSGNTAT LTISRVEAGD EADYYCQVWD SSSDVVFGGG |
| 121 | TKLTVLGQPK AAPSVTLPPP SSEELQANKA TLVCLISDFY PGAVTVAWKA DSSPVKAGVE |
| 181 | TTTPSKQSNN KYAASSYLSL TPEQWKSHRS YSCQVTHEGS TVEKTVAPTE CS |

What is claimed is:

1. Twelve protein biomarkers as related to breast cancer.

2. One or more of the biomarkers of claim 1, whereby up to 12 protein biomarkers in human blood identified as related to breast cancer are employed in a diagnostic assay for differentiating between patients having breast cancer, having benign breast disease or abnormalities, and normal controls. The method comprises:
collecting a biological sample from a patient having biopsy confirmed and histological staged breast cancer, a patient having a benign breast abnormality or disease, and a patient having no evidence of breast disease or breast abnormality,
determining the concentrations of up to 12 protein biomarkers identified as related to breast cancer and
determining whether or not the patient has breast cancer, based on a statistical analysis of the concentration in blood serum of the one or more of the selected 12 protein biomarkers.

3. One or more of the biomarkers of claim 1, whereby up to 12 protein biomarkers in human blood identified as related to breast cancer, and/or benign breast disease are employed in a screening assay for screening whether a patient has breast cancer. The method comprises:
collecting a biological sample from a patient,
determining the concentrations of up to 12 protein biomarkers identified as related to breast cancer and
determining whether or not the patient has breast cancer, based on a statistical analysis of the concentration in blood serum of one or more of the selected 12 protein biomarkers.

4. The method of claim 2, wherein the statistical analysis is an analysis of variance, a discriminant analysis, and/or a statistical plot such as a Box and Whiskers plot.

5. A biomarker of claim 1, wherein the biomarker is one of the following 12 biomarkers:
an inter-alpha-trypsin inhibitor heavy chain (H4) related protein, processing product of same, or one or more of the protein isoforms or post-synthetic modification variants of an inter alpha-alpha-trypsin inhibitor heavy chain (H4) related protein.

6. A biomarker of claim 1, wherein the biomarker is an immunoglobulin lambda chain protein, and/or processing product and/or one or more of the protein isoforms or post-synthetic modification variants of an immunoglobulin X chain protein.

7. A biomarker of claim 1, wherein the biomarker is an alpha-1-microglobulin protein, a processing product and/or one or more of the biomarker protein isoforms or post-synthetic modification variants of an alpha-1-microglobulin protein.

8. A biomarker of claim 1, wherein the biomarker is an Apolipoprotein A-I protein, processing product and/or one or more of the biomarker protein isoforms or post-synthetic modification variants of Apolipoprotein A-I.

9. A biomarker of claim 1, wherein the biomarker is an Apolipoprotein E protein, an Apolipoprotein E3 protein, processing product and/or one or more of the biomarker protein isoforms or post-synthetic modification variants of Apolipoprotein E or Apolipoprotein E3.

10. A biomarker of claim 1, wherein the biomarker is Complement C4 protein, a Complement C4A protein, a Complement C4A gamma chain protein, a processing product and/or one or more of the biomarker protein isoforms or post-synthetic modification variants of a Complement C4 protein, of a Complement C4A protein, and/or of a Complement C4A gamma chain protein.

11. A biomarker of claim 1, wherein the biomarker is a Serum Albumin protein, a processing product and/or one or more of the biomarker protein isoforms or post-synthetic modification variants of a Serum Albumin protein.

12. A biomarker of claim 1, wherein the biomarker is a Lectin P35 protein, a processing product and/or one or more of the biomarker protein isoforms or post-synthetic modification variants of a Lectin P35 protein.

13. A biomarker of claim 1, wherein the biomarker is a Transferrin protein, a processing product and/or one or more of the biomarker protein isoforms or post-synthetic modification variants of a Transferrin protein.
